(19) United States
(12) Patent Application Publication
(10) Pub. No.: US 2004/0186093 A1
(43) Pub. Date: Sep. 23, 2004

(54) Process for preparing sultams

(76) Inventors: Jaemoon Lee, Edison, NJ (US); David Askin, Warren, NJ (US); Mark S Jensen, Holmdel, NJ (US); Yong-Li Zhong, Woodbridge, NJ (US)

Correspondence Address: MERCK AND CO INC, P O BOX 2000, RAHWAY, NJ, 070650907

(21) Appl. No.: 10/486,526

(22) PCT Filed: Aug. 13, 2002
(86) PCT No.: PCT/US02/25666

(30) Foreign Application Priority Data
Aug. 17, 2001 (US) 60313375

(51) Int. Cl. A61K 31/554; A61K 31/54

(52) U.S. Cl. 514/211.01; 514/222.2; 544/1; 540/544

(57) ABSTRACT

The preparation of sultams is disclosed. In one embodiment (e.g. scheme (I)), an alkanesulfonyl halide is reacted with a haloalkylamine to obtain the corresponding N-(haloalkyl)alkanesulfonamide which is then cyclized in the presence of a deprotonating agent to give the sultam. The sultams are useful as intermediates in the preparation of naphthyridine carboxamide compounds which are HIV integrase inhibitors.

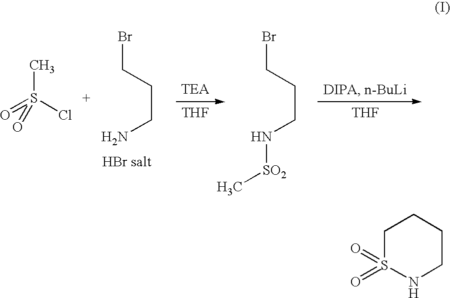

### FIELD OF THE INVENTION

The present invention is directed to a process for preparing alkanesultams. The sultams are useful as intermediates in the preparation of inhibitors of HIV integrase.

### BACKGROUND OF THE INVENTION

Alkanesultams are useful as intermediates in the preparation of compounds having pharmaceutical activity. The sultams can, for example, be used as intermediates in the preparation of certain 5-sulfonamido-8-hydroxy-1,6-naphthyridine-7-carboxamides which are inhibitors of HIV integrase and HIV replication and useful for preventing or treating HIV infection, delaying the onset of AIDS, and treating AIDS. Akanesultams have been prepared by the cyclization of aminoalkanesulfonyl chlorides. Dirscherl et al., Justus Liebigs Ann. Chem. 1954, 588: 200-204 discloses the preparation of 1,4-butanesultam by treating 4-aminobutanesulfonic acid with POCl₃ and then with PCl₅, and then evaporating the reaction mixture to dryness and neutralizing the residue with KOH. The sultam was then isolated by evaporating the neutralized product to dryness again, and then using repeated chloroform extractions and successive recrystallizations from diethyl ether. Dirscherl et al., Chem. Ber. 1956, 89: 393-395 discloses the preparation of 1,4-butanesultam by treatment of 4-aminobutanesulfonyl chloride dissolved in methanol with NaOH, wherein the sulfonyl chloride starting material was prepared by chlorination of a solution of bis-[δ-aminobutyl]-disulfide dihydrochloride in water. A similar procedure involving bromination was previously disclosed in Dirscherl et al., Justus Liebigs Ann. Chem. 1954, 588: 200-204.

Alkanesultams have also been prepared by the cyclization of chloroalkanesulfonamides. Ohashi et al., Bull Chem. Soc. Japan 1971, 44: 771-777 discloses the preparation of 1,4-butanesultam by refluxing N-t-butyl-δ-chlorobutanesulfonamide dissolved in EtOH with NaOH and then treating the resulting N-t-butylsultam with concentrated HCl in EtOH. The Ohashi et al. reference also discloses the preparation of 4-methylbutanesultam by treating N-t-butyl-δ-chloropentanesulfonamide with HCl, and then refluxing the treated product with NaOH in EtOH. Similar preparations for 4-ethylbutanesultam and 3-propylpropanesultam are also disclosed. The N-t-butylchloroalkanesulfonamide starting materials for these preparations were obtained via the photodecomposition of N-t-butyl-N-chloro-n-alkanesulfonamides, which in turn were prepared by chlorinating N-t-butyl-n-alkanesulfonamides (in addition to the Ohashi et al. reference, see Okara et al., Tet. Letters 1967, No. 17, 1629-1632).

White et al., J. Org. Chem. 1987, 52: 2162-2166 discloses the cyclization of 4-chlorobutanesulfonamide to 1,4-butanesultam by refluxing the 4-chlorobutane-sulfonamide with Na metal in EtOH. The 4-chlorobutane-sulfonamide reactant was itself prepared by (i) refluxing 4-chlorobutyl acetate in water with sodium sulfite, (ii) treating the mixture from (i) with concentrated HCl to afford sodium 4-hydroxybutane-1-sulfonate, (iii) chlorinating the sodium sulfonate salt of (ii) with PCl₅, and (iv) treating the resulting 4-chlorobutanesulfonyl chloride with excess ammonia to obtain 4-chlorobutanesulfonamide. The White et al. reference also discloses analogous preparations for 1,3-propanesultam and 1,5-pentanesultam.

Alkanesultams have also been prepared by cyclizing γ-aminocarbonyl sulfonates and γ-carbomethoxy sulfonamides and then reducing the resulting cyclic sulfimides. Morris et al., J. Org. Chem. 1991, 56: 3549-3556 discloses the cyclization of γ-(aminocarbonyl)benzene-propanesulfonate to dihydro-4-phenyl-2H-1,2-thiazin-3(4H)-one 1,1-dioxide by treatment with potassium tert-butoxide, followed by reduction of the thiazinone dioxide to the corresponding 4-phenyl-[1,2]-thiazinane 1,1-dioxide using NaCF₃CO₂BH₃. The Morris et al. reference also discloses the cyclization of certain 3-aryl-3-methoxycarbonylpropanesulfonamides to their corresponding sulfimides by treatment with sodium hydride or sodium methoxide, and then the reduction of the cyclic sulfimides to their corresponding 4-aryl-[1,2]-thiazinane 1,1dioxides using NaCF₃CO₂BH₃.

A disadvantage of the known methods for preparing sultams is that the reactants are not readily available commercially and are difficult to prepare in high yields, particularly on a large scale. For example, as described above, the 4-chlorobutanesulfonamide reactant cyclized in White et al. to afford butanesultam is obtained via a four-step synthesis starting with 4-chlorobutylacetate. The route to the 4-chlorobutanesulfonamide also involves the use of PCl₅, which is a corrosive, flammable, pungent reagent that requires careful handling. Another example is the route described in Ohashi et al. involving the cyclization of N-t-butylchloroalkyl-sulfonamide, which is obtained in a multi-step procedure that includes chlorination of the corresponding N-t-butyl-N-alkanesulfonamide, followed by the photodecomposition of the resulting N-t-butyl-N-chloro-n-alkanesulfonamide, and then isolation of the desired N-t-butylchloroalkylsulfonamide from the photoproduct mixture. Still another example is the 4-aminobutanesulfonyl chloride employed as the reactant in Discherl et al., which is prepared by treating 4-aminobutanesulfonic acid with PCl₅. While not disclosed in Discheral et al., the 4-aminobutanesulfonic acid also requires preparation; e.g., by reaction of a sultone with ammonia.

There is a need for a new method for preparing alkanesultams that employs a starting reactant which is either readily available commercially or which can be prepared in high yield in a minimum of steps from relatively simple starting materials.

### SUMMARY OF THE INVENTION

The present invention is directed to a process for preparing sultams which are useful as intermediates in the preparation of HIV integrase inhibitors. The present invention includes a process for preparing a compound of Formula (I):

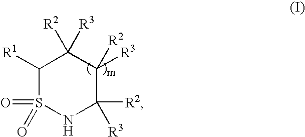

which comprises:

(B) treating a compound of Formula (II):

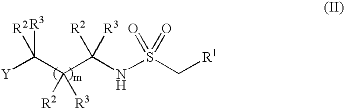

with a deprotonating agent in an aprotic solvent to obtain Compound I; wherein

Y is a leaving group without an active proton;

R¹ is H, C₁₋₆ alkyl or phenyl; wherein the phenyl is optionally substituted with one or more substituents (e.g., from 1 to 5 substituents, or from 1 to 4 substituents, or from 1 to 3 substituents; or is di-substituted; or is mono-substituted) each of which is halo or C₁₋₆ alkyl;

each R² is independently H or C₁₋₆ alkyl;

each R³ is independently H or C₁₋₆ alkyl; and

m is an integer equal to zero, 1 or 2.

An embodiment of the present invention is a process which comprises Step B as described above and further comprises:

(A) reacting a sulfonyl halide of Formula (III) or a sulfonyl anhyride of Formula (IV):

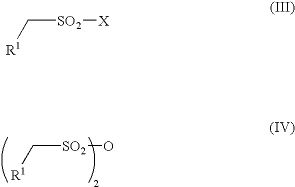

with an amine of Formula (V):

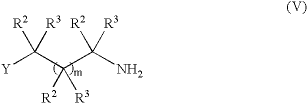

in an aprotic solvent to obtain Compound II; wherein X is halo.

The process of the invention represents a different approach to the preparation of sultams relative to known methods. While not wishing to be bound by any particular theory, the cyclization of Compound II in Step B of the present invention is believed to proceed via intramolecular dianion alkylation, wherein sufficient deprotonating agent is employed to deprotonate both the sulfonamido nitrogen and the methylene group alpha to the sulfonyl, followed by formation of the sultam ring by attack of the deprotonated methylene anion on the Y—CR²R³ carbon with displacement of the Y group. In sharp contrast, the known cyclizations typically involve formation of a cyclic sulfonamide by intramolecular nucleophilic substitution of a sulfonyl chloride with an amino group (see, e.g., the Dirscherl et al., Chem. Ber. reference in the Background) or by intramolecular nucleophilic substitution of an alkyl halide with a sulfonamide group (see, e.g., the White et al., J. Org. Chem. reference in the Background).

As noted in the Background of the Invention, known methods for making unsubstituted alkanesultams (e.g., 1,3-propanesultam, 1,4-butanesultam, and 1,5-pentanesultam) have the disadvantage of involving the cyclization of reactants that are neither readily available commercially nor easy to prepare in high yields. On the other hand, the reactants which are cyclized in accordance with the present invention to make unsubstituted alkanesultams can be prepared in high yields in one or two steps from relatively simple, commercially available starting materials. For example, reactants such as N-(3-halopropyl)alkane sulfonamides and N-(4-halobutyl)alkanesulfonamides can, in accordance with optional Step A of the invention as set forth above, be prepared by reacting an alkanesulfonyl halide (e.g., MsCl) with the corresponding haloalkylamine. The alkanesulfonyl halides are generally commercially available (e.g., MsCl), or they can be prepared by halogenating the corresponding sulfonic acids. The haloalkylamines are also commercially available.

### DETAILED DESCRIPTION OF THE INVENTION

The present invention includes the preparation of sultams of Formula (I) by the process set forth above in the Summary of the Invention. The sultams are useful as intermediates in the preparation of certain 5-sulfonamido-8-hydroxy-1,6-naphthyridine-7-carboxamides which are inhibitors of HIV integrase and HIV replication and useful for preventing or treating HIV infection, delaying the onset of AIDS, and treating AIDS.

An embodiment of the present invention is the process comprising Step B as originally defined above and optionally further comprising Step A as defined above, wherein R¹ in Compounds I and II (and optionally also in Compound III or Compound IV) is H, C₁₋₄ alkyl or phenyl. In an aspect of this embodiment, R¹ is H, methyl, or phenyl. In another aspect of this embodiment, R¹ is H.

Other embodiments of the process of the invention include the process as originally defined above or as defined in any one of the preceding embodiments or aspects, wherein each R² is independently H, methyl, or ethyl; and each R³ is independently H, methyl, or ethyl. In an aspect of each of these embodiments, each R² is H, and each R³ is independently H, methyl, or ethyl. In another aspect of each of these embodiments, each R² is H and each R³ is H.

Y can be any leaving group which does not contain an active proton, the presence of which could interfere with the desired dianion alkylation. Y is suitably a weakly nucleophilic group. Exemplary Y groups include halo (e.g., chloro or bromo), arylsulfonato (e.g. benzenesulfonato), and phosphato (e.g., diisopropylphosphato). Embodiments of the process of the invention include the process as originally defined above or as defined in any one of the preceding embodiments or aspects in which Y is halo, —OSO₂R^{a}, or —OP(═O)(OR^{b})₂; wherein R^{a} is phenyl which is optionally substituted with one or more substituents (e.g., from 1 to 5 substituents, or from 1 to 4 substituents, or from 1 to 3 substituents; or is di-substituted; or is mono-substituted) each of which is fluoro, chloro, methoxy, or t-butyl; and each R^{b} is independently a C₁₋₆ alkyl. In an aspect of each of these embodiments, Y is halo. In other aspects of each of these embodiments, Y is chloro or bromo; or is bromo; or is chloro.

Other embodiments of the process of the invention include the process as originally defined above or as defined in any one of the preceding embodiments or aspects, wherein m is zero or 1; or m is zero; or m is 1; or m is 2.

It is understood that m=zero means that a direct single bond exists between the ring carbon atoms that would otherwise have been indirectly attached to each other via the —(CR²R³)ₘ— moiety. For example, when m is zero, the sultam I is:

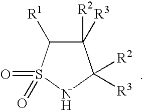

Other embodiments of the process of the invention include any one of the preceding embodiments or aspects which incorporates Step A, wherein X is chloro, bromo or iodo; or X is chloro or bromo; or X is chloro; or X is bromo.

The solvent employed in the cyclization reaction of Step B can be any organic compound which under the reaction conditions employed is in the liquid phase, is chemically inert, and will dissolve, suspend, and/or disperse Compound II and the deprotonating agent so as to permit the reaction to proceed. The solvent is suitably an aprotic solvent. Suitable solvents include hydrocarbons (i.e., aliphatic, alicyclic and aromatic hydrocarbons) and ethers (i.e., mono-, di- and poly-ethers). Exemplary solvents include pentane, hexane, cyclohexane, benzene, toluene, xylenes, THF, MTBE, ethyl ether, dioxane, DME, anisole, and phenetole.

In one embodiment, the solvent employed in Step B is selected from the group consisting of C₅-C₁₀ linear and branched alkanes, C₅-C₁₀ cycloalkanes, C₆-C₁₄ aromatic hydrocarbons, di-C₁-C₆ alkyl ethers, C₁-C₆ linear and branched alkanes substituted with two —O—C₁-C₆ alkyl groups (which are the same or different), C₄-C₈ cyclic ethers and diethers, and C₆-C₁₀ aromatic ethers. In another embodiment, the solvent employed in Step B is an ether. In an aspect of the preceding embodiment, the ether is THF, MTBE, or DME.

Step B is suitably conducted at a temperature in the range of from about −78 to about 50° C., and is typically conducted at a temperature in the range of from about −78 to about 25° C. (e.g., from about −78 to about 10° C., or from about −60 to about 10° C.). In one embodiment, the temperature is in the range of from about −45 to about 10° C. (e.g., from about −30 to about 0° C.).

The deprotonating agent in Step B can be any base having sufficient strength under the selected reaction conditions to deprotonate Compound II to form a dianion. Suitable deprotonating agents include those selected from the group consisting of alkali metal salts and alkaline earth metal salts of di-C₁-C₉ alkylamines and C₄-C₉ cyclic secondary amines, alkali metal salts and alkaline earth metal salts of bis(tri-C₁-C₄ alkylsilyl)amines, alkali metal hydrides, alkali metal amides, C₁-C₁₀ alkyllithiums, C₆-C₁₀ aryllithiums, C₁-C₆ alkylmagnesium halides, and C₆-C₁₀ arylmagnesium halides. In one embodiment, the deprotonating agent is an alkali metal salt of a di-C₁-C₉ alkylamine or a C₄-C₉ cyclic secondary amine.

Exemplary deprotonating agents include methyllithium, n-butyllithium, tert-butyllithium, sec-butyllithium, phenyllithium, phenyl sodium, phenyl potassium, lithium amide, sodium amide, potassium amide, lithium piperidide, lithium tetramethylpiperidide, lithium diisopropylamide, lithium diethylamide, lithium dicyclohexylamide, sodium hexamethyldisilazide, lithium hexamethyldisilazide, sodium hydride, potassium hydride, ethylmagnesium chloride, isopropylmagnesium chloride, phenylmagnesium chloride, ethylmagnesium bromide, isopropylmagnesium bromide, and phenylmagnesium bromide.

The deprotonating agent can be employed in Step B in any proportion which will result in the formation of at least some of Compound I. Typically, however, the deprotonating agent is employed in an amount sufficient to optimize conversion of Compound II. In one embodiment, the deprotonating agent is employed in Step B in an amount of from about 1.1 to about 10 equivalents per equivalent of Compound II. In another embodiment, the deprotonating agent is employed in an amount of from about 1.3 to about 5 equivalents per equivalent of Compound II. In still another embodiment, the amount of deprotonating agent employed in Step B is from about 1.8 to about 3 equivalents (e.g., from about 2 to about 2.5 equivalents) per equivalent of Compound II. In yet another embodiment, the amount of deprotonating agent employed in Step B is from about 2.1 to about 2.3 equivalents (e.g., about 2.2 equivalents) per equivalent of Compound II.

The Step B reaction can be conducted by dissolving, suspending or dispersing Compound II in the aprotic solvent in a suitable reaction vessel, bringing the mixture to the desired reaction temperature (note: the mixture is typically cooled since the reaction is most often conducted at a temperature below room temperature), and then slowly adding the deprotonating agent (e.g., an alkali metal amide) while maintaining the mixture at the desired reaction temperature until the reaction is complete or the desired degree of conversion of Compound II is achieved. The reaction is generally conducted under an inert atmosphere (e.g., nitrogen or argon gas). The reaction time can vary widely depending upon, inter alia, the reaction temperature and the choice and relative amounts of Compound II and the deprotonating agent, but the reaction time is typically in the range of from about 1 to about 24 hours. The reaction can be quenched by addition of an aqueous salt solution, and the sultam recovered from the organic phase.

Optional Step A of the process of the invention as set forth above in the Summary of the Invention is directed to the preparation of Compound A, which is then cyclized in Step B to obtain sultam I. The solvent used in Step A can be any organic compound which under the reaction conditions employed is in the liquid phase, is chemically inert, and will dissolve, suspend, and/or disperse the reactants so as to permit the reaction to proceed. Suitable solvents includes hydrocarbons, halogenated hydrocarbons, and ethers. Exemplary solvents include pentane, hexane, cyclohexane, benzene, toluene, xylenes, carbon tetrachloride, chloroform, methylene chloride, 1,2-dichloroethane, 1,1,2-trichloroethane, 1,1,2,2-tetrachloroethane, THF, MTBE, ethyl ether, dioxane, DME, anisole, and phenetole.

In one embodiment, the solvent in Step A is selected from the group consisting of C₃-C₁₀ linear and branched alkanes, halogenated C₁-C₁₀ linear and branched alkanes, C₅-C₁₀ cycloalkanes, C₆-C₁₄ aromatic hydrocarbons, halogenated C₆-C₁₄ aromatic hydrocarbons, di-C₁-C₆ alkyl ethers, C₁-C₆ linear and branched alkanes substituted with two —O—C₁-C₆ alkyl groups (which are the same or different), C₄-C₈ cyclic ethers and diethers, and C₆-C₁₀ aromatic ethers. In another embodiment, the solvent employed in Step B is an ether. In an aspect of the preceding embodiment, the ether is THF, MTBE, or DME. In still another embodiment, the solvent employed in Step A is the same as the solvent employed in Step B. In an aspect of the preceding embodiment, the solvent in Steps B and A is the same and is an ether (e.g., THF or DME).

Step A is suitably conducted at a temperature in the range of from about −78 to about 50° C., and is typically conducted at a temperature in the range of from about −78 to about 30° C. (e.g., from about −30 to about 30° C.). In one embodiment, the temperature is in the range of from about −20 to about 25.

Sulfonyl halide III or sulfonyl anhydride IV can be employed in Step A in any proportion with respect to amine V which will result in the formation of at least some of Compound II. Typically, however, the reactants are employed in proportions which can optimize conversion of at least one of the reactants. In one embodiment, sulfonyl halide III or sulfonyl anhydride IV is employed in Step A in an amount in the range from about 0.2 to about 5 equivalents (e.g., from about 0.5 to about 5 equivalents) per equivalent of amine V. In another embodiment, sulfonyl halide III or sulfonyl anhydride IV is employed in Step A in an amount in the range from about 0.8 to about 2 equivalents per equivalent of amine V. In still another embodiment, sulfonyl halide III or sulfonyl anhydride IV is employed in Step A in an amount in the range from about 0.9 to about 1.5 equivalents (e.g., from about 1 to about 1.5 equivalents) per equivalent of amine V. In still another embodiment, sulfonyl halide III or sulfonyl anhydride IV is employed in Step A in an amount in the range from about 1.1 to about 1.3 equivalents per equivalent of amine V.

The Step A reaction can be conducted by slowly adding a solution of the sulfonyl halide III (or the sulfonyl anhydride IV) in the selected solvent (e.g., an ether such as THF) to a suitable reaction vessel containing a cooled mixture of amine V and the solvent while maintaining the temperature. A suitable base (e.g., a tertiary amine such as TEA) can be added concurrently with addition of III (or IV) to neutralize the acidic HX byproduct generated by sulfonamide formation. In addition, if a salt of amine V is employed (e.g., an acid salt of a haloalkylamine) in the reaction, addition of base can promote dissolution of the salt. After the addition of III or IV and the base, the mixture is brought to and maintained at reaction temperature until the reaction is complete or the desired degree of conversion of amine V is achieved. The reaction time can vary widely depending upon, inter alia, the reaction temperature and the choice and relative amounts of Compound III or IV, base, and amine V, but the reaction time is typically in the range of from about 1 to about 12 hours. The sulfonamide product II can be isolated (alternatively referred to herein as recovered) by conventional means. In one embodiment, Compound II is not isolated at the conclusion of Step A, but instead the reaction mixture containing Compound II is employed directly in Step B. In an aspect of this embodiment, the solution of Compound II resulting from Step A is prepared for use in Step B by removing unwanted byproducts therefrom (e.g., removal of suspended solids by filtration) and then transferring the solution to a suitable reaction vessel to conduct Step B by addition of deprotonating agent in the manner described earlier. In another aspect of this embodiment, Step B is conducted in the same reaction vessel employed in Step A (i.e., a “one pot” synthesis of the sultam).

The present invention also includes a process for preparing 1,4-butanesultam 4:

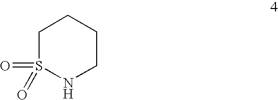

which comprises:

(bb) treating a compound of Formula (IIa):

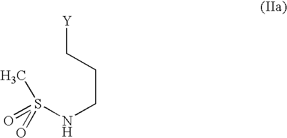

with a deprotonating agent in an aprotic solvent to obtain Compound 4; wherein Y is a leaving group without an active proton.

An embodiment of the process for preparing Compound 4 is a process which comprises Step (bb) as set forth above and further comprises:

(aa) reacting a methanesulfonyl halide of Formula (IIIa) or methanesulfonic anhydride of Formula (IVa):

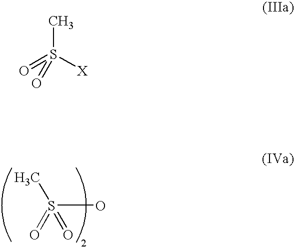

with an amine of Formula (Va):

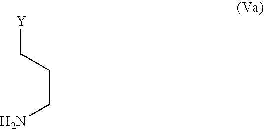

in an aprotic solvent to obtain Compound (IIa); wherein X is halo.

All solvents, agents, reaction conditions, and relative amounts of reactants and reagents described above for use in Steps A and B above can be used in Steps (aa) and (bb). In particular, additional embodiments of the process for preparing Compound 4 include the process comprising Step (bb) as first described and optionally further comprising Step (aa) as just described, and incorporating any one or more of the following features of Step (bb):

Y is halo, —OSO₂R^{a}, or —OP(═O)(OR^{b})₂, wherein R^{a} and R^{b} are as defined above; or is halo; or is chloro or bromo; or is chloro; or is bromo;

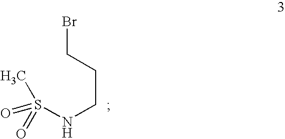

the treatment in Step (bb) is conducted at a temperature in the range of from about −78 to about 10° C., or from about 45 to about 10° C.;

the deprotonating agent is an alkali metal salt of a di-C₁-C₉ alkylamine or a C₄-C₉ cyclic secondary amine;

the deprotonating agent is LDA or is nBuLi with DIPA such that LDA is formed in situ for use in Step (bb);

the aprotic solvent in Step (bb) is an ether (e.g., THF, DME or MTBE); and

the deprotonating agent in Step (bb) is employed in an amount in the range from about 1.8 to about 3 equivalents (or from about 2 to about 2.5 equivalents) per equivalent of Compound IIa.

Additional embodiments of the process for preparing Compound 4 include the process comprising Step (bb) as first described above optionally incorporating any one or more of the just-described features, and further comprising Step (aa) including any one or more of the following features:

X is chloro or bromo; or is chloro; or is bromo;

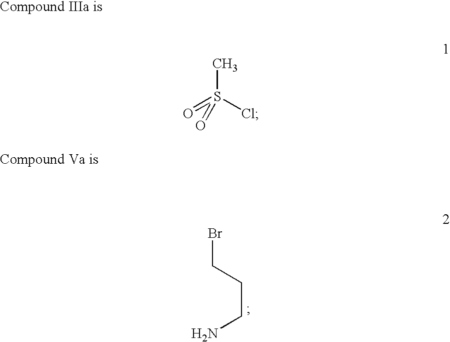

the reaction in Step (aa) is conducted at a temperature in the range of from about −78 to about 30° C., or from about −30 to about 30° C.;

the aprotic solvent in Step (aa) is an ether (e.g., THF, DME or MTBE); and

methanesulfonyl halide Ma or methanesulfonic anhydride IVa is employed in Step (aa) in an amount in the range from about 0.5 to about 5 equivalents (e.g., from about 0.9 to about 1.5 equivalents) per equivalent of amine Va.

Still further embodiments of the process for preparing Compound 4 include a process comprising Step (aa) and Step (bb), either of which is as first described or is as described in any one of the preceding embodiments, wherein the aprotic solvent in both Steps (aa) and (bb) is the same; and the process is conducted without isolating Compound IIa from the reaction mixture at the conclusion of Step (aa). In an aspect of these embodiments, Steps (aa) and (bb) are conducted in the same reaction vessel (i.e., a one-pot process).

The invention is also a process for preparing a compound of Formula I)

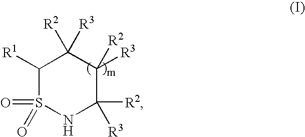

which comprises deblocking a compound of Formula (VI):

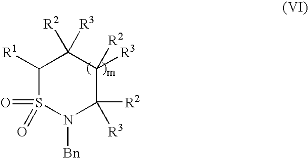

with a deblocking agent, wherein

R¹ is H, C₁₋₆ alkyl or phenyl; wherein the phenyl is optionally substituted with one or more substituents each of which is halo or C₁₋₆ alkyl;

each R² is independently H or C₁₋₆ alkyl;

each R³ is independently H or C₁₋₆ alkyl; and

m is an integer equal to zero, 1 or 2.

An embodiment of the process for deblocking the compound of Formula (VI) further comprises substituting a compound of Formula (VII)

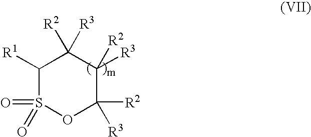

with a substituting agent selected from the group consisting of arylamine and alkylamine to form a slurry product, cooling the slurry product, and cyclizing the slurry product with a cyclizing agent. In an embodiment of the substitution, the substituting agent is benzylamine. In an embodiment of the cyclizing step, the cyclizing agent is phosphorus oxychloride. Preferably, m is 1, R¹ is hydrogen, R² is hydrogen and R³ is hydrogen.

Still other embodiments of the present invention include any of the processes as originally defined and described above and any embodiments or aspects thereof as heretofore defined, further comprising isolating (alternatively referred to as recovering) the compound of interest (e.g., Compound I) from the reaction medium.

If desired, the progress of the reaction in any of the above-described chemical reactions can be followed by monitoring the disappearance of a reactant and/or the appearance of the product using TLC, HPLC, NMR, or GC.

As used herein, the term “C₁-C₆ alkyl” (which may alternatively be referred to herein “C₁₋₆ alkyl”) as means linear or branched chain alkyl groups having from 1 to 6 carbon atoms and includes all of the hexyl alkyl and pentyl alkyl isomers as well as n-, iso-, sec- and t-butyl, n- and isopropyl, ethyl and methyl. “C₁-C₄ alkyl” (or “C₁₋₄ alkyl”) means n-, iso-, sec- and t-butyl, n- and isopropyl, ethyl and methyl.

The term “halogen” (or “halo”) refers to fluorine, chlorine, bromine and iodine (alternatively, fluoro, chloro, bromo, and iodo).

The term “haloalkyl” means a linear or branched alkyl group with one or more halogen substituents.

The term “aryl” refers herein to an aromatic carbocyclic group such as phenyl, naphthyl, anthryl, or phenanthryl.

The term “substituted” (which appears in such expressions as “substituted with one or more of . . . ”) includes mono- and poly-substitution by a named substituent to the extent such single and multiple substitution is chemically allowed and results in a chemically stable compound.

Combinations of substituents and/or variables are permitted only to the extent such combinations result in chemically stable compounds.

When any variable (e.g., R², R³, and R^{b}) occurs more than one time in any constituent or in any formula, its definition on each occurrence is independent of its definition at very other occurrence.

Abbreviations used in the instant specification include the following:

AIDS=acquired immunodeficiency syndrome

ARC=AIDS related complex

Bu=butyl

DME=1,2-dimethoxyethane

DMF=N,N-dimethylformamide

DIPA=diisopropylamine

EDTA=ethylenediamine tetraacetic acid

Et=ethyl

EtOAc=ethyl acetate

EtOH=ethanol

g=gram(s)

h=hour(s)

HIV=human immunodeficiency virus

HPLC =high-performance liquid chromatography

Me=methyl

MeCN=acetonitrile

MeOH=methanol

min=minute(s)

Ms=mesyl (methanesulfonyl)

MTBE=methyl t-butyl ether

NMR=nuclear magnetic resonance

TEA=triethylamine

THF=tetrahydrofuran

The following examples serve only to illustrate the invention and its practice. The examples are not to be construed as limitations on the scope or spirit of the invention.

### EXAMPLE 1

### Preparation of 1,4-butanesultam

### Step 1: Preparation of N-(3-bromoprop-1-yl)methanesulfonamide

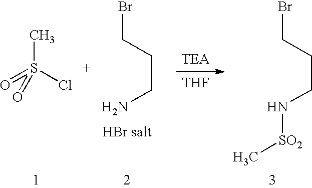

The HBr salt of 3-bromopropylamine (2 (103.3 g, 469 mmol) and THF (300 mL) were placed in 3-neck round bottomed flask (2 L), and the resulting slurry was cooled to about 5° C. with an ice-methanol bath (−10° C.). Triethylamine (132 mL, 943 mmol) was added via dropping funnel over 30 min. During this time, the solution of methanesulfonyl chloride (1, 37.5 ml, 485mmol) in 90 mL of THF was slowly added via a dropping funnel over a period of about 30 min while keeping the internal temp between about 5-10° C. The addition of TEA was slightly faster than that of MsCl. After completing the addition of TEA and MsCl, the slurry was aged for 1 h at this temperature (about 5-10° C.), after which the reaction mixture was warmed to room temperature and stirred for an additional 1.5 h. MTBE (600 mL) was charged and stirred for 10 min prior to filtering over sintered glass. After rinsing the cake with MTBE (200 mL), the filtrates were washed with saturated NaHCO₃ (300 mL) and then with brine (300 mL). Evaporation of the organic layer followed by drying under vacuum afforded 96 g (95% yield) of the bromo-sulfonamnide 3. Methanol was used for the azeotropic removal of water. After drying, ¹H NMR showed the product was free of methanol. This crude material was used for next step without further purification.

¹H NMR(CDCl₃, 400 mHz) δ 4.55 (br s, 1H), 3.50 (t, 2H), 3,35 (m, 2H), 3.30 (s, 3H), 2.15 (q, 2H).

### Step 2: Preparation of the Butanesultam

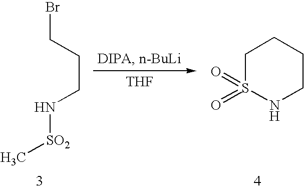

THF (1260 mL) and the bromo-sulfonamide prepared in Step 1 (90 g, 417 mmol) were placed in a 2 L, 3-neck round bottomed flask and cooled to −20° C. Diisopropylamine (14.6 mL, 104 mmol) and 1,10-phenanthroline (90 mg) were then added, followed by the slow addition of n-BuLi solution (1.6 M in hexane solution, 546 mL, 837 mmol) via a dropping funnel, while maintaining the internal temperature in the range of from −20° C. to −10° C. The addition took 45 min. with a cooling bath temperature of about −30° C. The resulting solution was aged at −10° C. to 0° C. for 3 h. The progress of the reaction was followed by monitoring the disappearance of starting material using ¹H NMR. Following completion of the reaction, the reaction was quenched by the sequential addition of saturated NH₄Cl (300 mL) at 0° C. and brine (300 mL). The resulting phases were separated, and the aqueous layer extracted with 500 mL of ethyl acetate. The organic layers were concentrated and the solvent switched to ethyl acetate. The volume of ethyl acetate was adjusted to about 300 mL at about 40-45° C., and then heptane (500 mL) was added with stirring. The slurry was cooled to 0° C. and aged for 30 min. The crystalline solid was then separated by filtration, and dried under vacuum oven for 16 h to give 41 g of the desired δ-sultam 4 as a yellow solid (72% yield).

¹H NMR (CDCl₃, 400 mHz) δ 4.36 (br s, 1H), 3.45 (m, 2H), 3.10 (m, 2H), 2.24 (m 2H), 1.64 (m, 2H).

### EXAMPLE 2

### Preparation of 1,4-Butanesultam

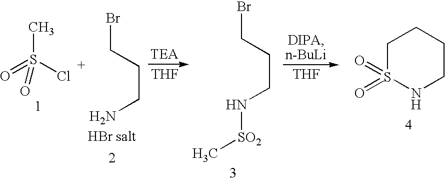

| | | | | | Den- | |
|---|---|---|---|---|---|---|
| | Weight | FW | Moles | Equiv. | sity | Volume |
| MsCl (1) | 2.36 Kg | 114.55 | 20.6 | 1.03 | 1.480 | 1.59 L |
| 3-bromo- | 4.40 Kg | 220 | 20.0 | 1.00 | | |
| propyl-amine | | | | | | |
| (2) HBr salt | | | | | | |
| TEA | 4.07 Kg | 101.19 | 40.2 | 2.01 | 0.726 | 5.60 L |
| THF | | | | | 43 + 4 + 8 = | |
| | | | | | 55 L | |
| DIPA | 481 g | 101.19 | 4.75 | 0.25 | 0.722 | 666 mL |
| 1,10-Phenan- | 4.11 g | 180.21 | | | | |
| throline | | | | | | |
| n-BuLi, | | | | | | |
| 1.6 M in hexane | | | | | | |

The 3-bromopropylamine-HBr salt ( and THF (43 L) were placed in a 72 L round-bottomed-flask under N₂ and the resulting slurry was cooled to 0° C. Two dropping funnels were fitted to the flask. One was charged with the TEA and the other with a solution of the MsCl (1 and THF (4 L). The contents of the addition funnels were added at roughly the same rate (the TEA was added slightly faster than the MsCl) while maintaining an internal reaction temperature below 10° C. The addition required 2 h. The resulting white suspension was warmed to 23° C and aged for 1 h. The suspended solids (a mixture of TEA-HBr and TEA-HCl) were removed by filtration through a dry frit. The cake was washed with THF (8 L). The combined filtrate and cake-rinse, a THF solution of 3, was collected in a 100 L round-bottomed-flask under N₂. To the solution of 3 was added the 1,10-phenanthroline and the DIPA and the resulting solution was cooled to −30° C. The n-BuLi was added over about 4 h maintaining the internal temperature below −20° C. After 1.25 eq of the n-BuLi was added the reaction mixture became deep brown and the color remained as the addition was completed. The reaction mixture was warmed to 0° C. over 3 h. A small aliquot was removed, and partitioned between saturated NH₄Cl and EtOAc. The EtOAc was evaporated and the residue examined by ¹H NMR to confirm consumption of 3 and conversion to 4. To the reaction mixture at 0° C. was added saturated aqueous NH₄Cl (12 L, the first 1L slowly, a heat kick to 6° C. was observed) and then brine (12 L). The phases were partitioned and the aqueous phase was extracted with EtOAc (20 L). The organic phases were combined, washed with brine (4 L) and then concentrated under vacuum to about 12 L. The solvent was switched to EtOAc (20 L used) maintaining a volume of 12 L. After the solvent switch, a yellow slurry resulted. n-Heptane (20 L) was added with stirring and the slurry was cooled to 5° C. After a 1 h age the solids were collected on a frit and rinsed with cold (5° C.) 3:5 EtOAc/n-heptane. The wet cake was dried for 24 h under a stream of dry N₂ to provide 1.44 Kg (53% from @2 of sultam 4 as a crystalline yellow solid.

### EXAMPLE 3

### Preparation of 5-(1,1-dioxido-1,2-thiazinan-2-yl)-N-(4-fluorobenzyl)-8-hydroxy-1,6-naphthyridine-7-carboxamide from methyl 5-bromo-8-hydroxy-1,6-naphthyridine-7-carboxylate

### Step 1: 5-Bromo-8-hydroxy-1,6-naphthyridine-7-carboxylic acid methyl ester

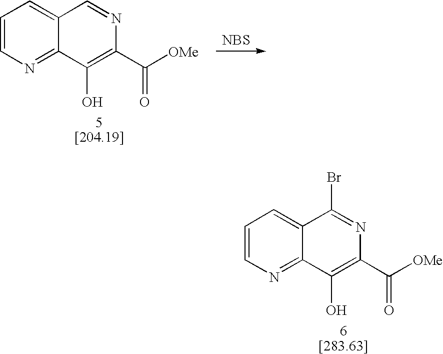

N-bromosuccinimide (7.83 g, 44.0 mmol) was added to a solution of 8-hydroxy-1,6-naphthyridine-7-carboxylic acid methyl ester (5, 8.17 g, 40.0 mmol) in chloroform (32 mL) over 20 min maintaining the temperature at 20-50° C. and the mixture was aged for 30 min at 50° C. The mixture became a thick, stirrable slurry and HPLC analysis indicated <2% starting material remaining. The mixture was cooled to 30° C. over 15 min. MeOH (64 mL) was added over 30 min then a 1:1 mixture of MeOH-water (64 mL) was added over 30 min. The mixture was cooled to −40° C. over 30 min and aged at −40° C. for 30 min. The cold mixture was filtered and the solid was washed with 1:1 MeOH:water (100 mL) at 10-20° C. The off white crystalline solid was dried under a stream of nitrogen to provide 10.48 g (93% yield) of 5-bromo-8-hydroxy-1,6-naphthyridine-7-carboxylic acid methyl ester (6).

HPLC retention times: 5=2.2 min, 6=6.0 min, HPLC conditions: 150×4.6 mm ACE 3 C18 column, isocratic elution with 30% MECN in 0.025% aq H₃PO₄ at 1 mL/min, 25° C. with detection at 254 nm;

HPLC retention times: 5=1.8 min, 6=3.1 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 6 (CDCl₃, 100 MHz): 169.7, 156.3, 154.5, 143.9, 137.1, 132.4, 128.0, 126.1, 124.2, 53.4.

### Step 2: 5-Bromo-8-(4-toluenesulfonyloxy)-1,6-naphthyridin-7-carboxylic acid methyl ester

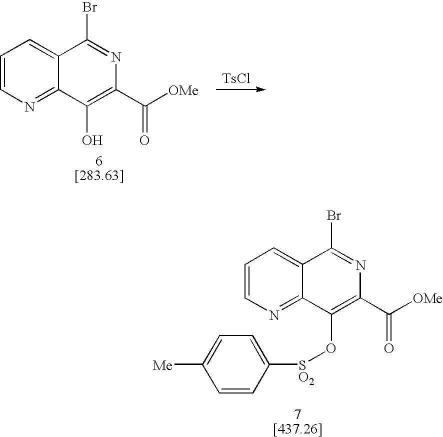

Triethylamine (0.759 g, 7.50 mmol) was added to a suspension of 5-bromo-8-hydroxy-1,6-naphthyridine-7-carboxylic acid methyl ester (§, 1.415 g, 5.000 mmol) in chloroform (5 mL) over 5 min maintaining the temperature at 20-50° C. to give a yellow suspension. p-Toluenesulfonyl chloride (1.15 g, 6.00 mmol) was added over 5 min maintaining the temperature at 206-40° C. to give a yellow solution. The mixture was aged at 40° C. for 2 h during which a crystalline solid precipitated out of the mixture and the color faded (HPLC analysis indicated <0.5% starting material remaining). The mixture was cooled to 20° C. over 15 min. MeOH (10 mL) was added over 30 min then a 1:1 mixture of MeOH:water (10 mL) was added over 30 min. The mixture was cooled to 40° C. over 30 min and aged at −40° C. for 30 min. The cold mixture was filtered and the solid was washed with 1:1 MeOH:water (10 mL), MeOH (5 mL), MTBE (10 mL) and hexanes (10 mL) all at 10-20° C. The off-white crystalline solid was dried under a stream of nitrogen to provide 2.112 g (97% yield) of 5-bromo-8-(p-toluenesulfonyloxy)-1,6-naphthyridine-7-carboxylic acid methyl ester (7).

HPLC retention times: 6=3.1 min, 7=12.4 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 7 (d6-DMSO, 100 MHz): 163.2, 157.0, 146.5, 145.8, 141.9, 141.3, 139.2, 137.2, 132.3, 130.4, 129.0, 127.6, 127.1, 53.3, 21.7.

### Step 3: 5-(1,1-Dioxido-1,2-thiazinan-2-yl)-8-(4-toluenesulfonyloxy)-1,6-naphthyridine-7-carboxylic acid methyl ester.

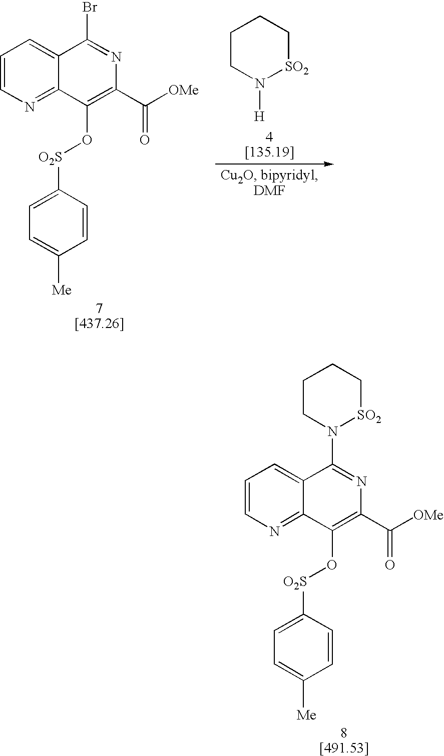

A mixture of 5-bromo-8-p-toluenesulfonyloxy)-1,6-naphthyridine-7-carboxylic acid methyl ester (7, 2.186 g, 5.000 mmol), 1,4-butane sultam (4, 811 mg, 6.00 mmol), copper (I) oxide (858 mg, 6.00 mmol, <5 micron), 2,2′-bipyridyl (937 mg, 6.00 mmol) and DMF (10 mL) was degassed by stirring under a stream of nitrogen for 1 min and heated to 120° C. for 4 h. The brown suspension became a dark red solution with a small amount of undissolved copper (I) oxide remaining (HPLC analysis indicated <0.5% starting material remaining). The mixture was diluted with chloroform (10 mL), Solkaflok (200 mg) was added and the resulting mixture was filtered through a plug of Solkaflok. The plug was washed with chloroform (10 mL) and the combined filtrates were stirred vigorously with a solution of EDTA disodium salt dihydrate (3.8 g, 10.2 mmol) in water (40 mL) while air was slowly bubbled in for 40 min. The upper aqueous phase became turquoise while the lower organic phase became yellow. The organic phase was washed with a solution of EDTA disodium salt (1.9 g, 5.1 mmol) in water (30 mL) and a solution of sodium bisulfate monohydrate (0.87 g, 6.3 mmol) in water (30 mL). Each of the above three aqueous phases was back extracted sequentially with one portion of chloroform (15 mL). The organic phases were dried over sodium sulfate and filtered. The dried organic extracts were concentrated and solvent switched to a final volume of 15 mL MeOH using a total of 30 mL MeOH for the switch at atmospheric pressure. Product crystallized during the solvent switch. The resulting slurry was cooled to 0° C. over 30 min and aged at 0° C. for 30 min. The slurry was filtered cold and the solid was washed with MeOH (15 mL). The off white solid was dried under a stream of nitrogen to provide 1.910 g (78%) of 5-(N-1,4-butanesultam)-8-(p-toluenesulfonyloxy)-1,6-naphthyridine-7-carboxylic acid methyl ester (8).

HPLC retention times: 7=12.4 min, 8=10.3 min, DMF=1.3 min, Bipy=1.5 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 8 (CDCl₃, 100 MHz): 164.2, 155.3, 151.9, 146.7, 145.4, 141.2, 137.8, 135.3, 133.6, 129.6, 128.9, 125.4, 124.3, 53.4, 52.9, 48.7, 24.2, 22.0, 21.7.

### Step 4: 5-(1,1-Dioxido-1,2-thiazinan-2-yl)-8-hydroxy-1,6-naphthyridine-7-carboxylic acid methyl ester.

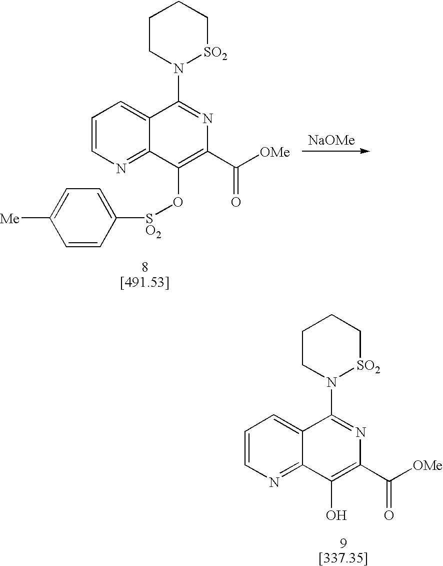

5-(N-1,4-butanesultam)-8-(p-toluenesulfonyloxy)-1,6-naphthyridine-7-carboxylic acid methyl ester (8 1.597 g, 3.250 mmol) was dissolved in DMF (3.25 mL) at 40° C. and transferred to a solution of 0.5M NaOMe in MeOH (16.25 mL, 8.125 mmol) over ca 1-2 min at 20-25° C. The resulting yellow homogenous mixture was heated to 50° C. and aged for 5 min (HPLC analysis indicated <0.5% starting material remaining). Mixture was cooled to 25° C. over 15 min and aged at 25° C. for 15 min during which a yellow crystalline precipitate was deposited. Acetic acid (390 mg, 6.50 mmol) was added over 1 min (yellow color faded) then water (32.5 mL) was added over 15 min at 25° C. The slurry was aged for 30 min 25° C. and filtered. The filter cake was washed with 1:1 MeOH:water (32.5 mL) and then with 1:1 MTBE:hexanes (8 mL). The filter cake was dried under a stream of nitrogen to provide 1.064 g (97%) of 5-(N-1,4-butanesultam)-8-hydroxy-1,6-naphthyridine-7-carboxylic acid methyl ester (9) as an off white crystalline solid.

HPLC retention times: 8=10.3 min, 9=2.9 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 9 (d6-DMSO, 100 MHz): 167.8, 154.4, 153.5, 143.9, 143.7, 135.2, 125.9, 125.2, 124.4, 53.2, 53.1, 49.1, 24.4, 21.9.

Step 5: 5-(1,1-Dioxido-1,2-thiazinan-2-yl)-N-(4-fluorobenzyl)-8-hydroxy-1,6-naphthyridine-7-carboxamide, monoethanolate.

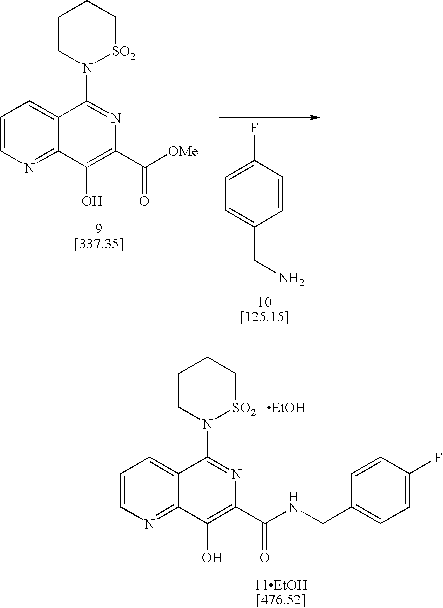

A suspension of 5-(N-1,4-butanesultam)-8-hydroxy-1,6-naphthyridine-7-carboxylic acid methyl ester (9, 1.012 g, 3.00 mmol) and 4-fluorobenzylamine (10, 1.314 g, 10.5 mmol) in EtOH (9.0 mL) was heated to 75-77° C. for 2 h during which the mixture became a yellow homogeneous solution (HPLC analysis indicated <0.5% starting material remaining). Acetic acid (0.630 mg, 10.5 mmol) was added over 1 min (yellow color faded) then water (9.0 mL) was added over 10 min at 75° C. An off white crystalline solid began to precipitate near the end of addition of the water. The slurry was cooled to 0° C. over 30 min then aged for 30 min at 0° C. and filtered. The filter cake was washed with 5% HOAc in 1:1 EtOH:water (5 mL) then with 1:1 EtOH:water (10 mL) and then with EtOH (5 mL). The filter cake was dried under a stream of nitrogen to provide 1.343 g (94%) of the monoethanolate of 5-(N-1,4-butanesultam)-N-(4-fluorobenzyl)-8-hydroxy-1,6-naphthyridine-7-carboxamide (11) as an off white crystalline solid.

HPLC retention times: 9=2.9 min, 11=6.7 min, 10=1.4 min, impurity present in 10=4.3 min, HPLC conditions: 150×4.6 mm ACE 3 C18 column, isocratic elution with 46% MeCN in 0.025% aq H₃PO₄ at 1 mL/min, 25° C with detection at 254 nm;

HPLC retention time: 9=10.9 min, HPLC conditions: 150×4.6 mm ACE 3 C18 column, isocratic elution with 24% MeCN in 0.025% aq H₃PO₄ at 1 mUmin, 25° C. with detection at 254 nm.

¹H NMR (d6-DMSO, 400 MHz): 9.25 (t, J=6.4, 1H), 9.16 (d, J=8.4, 1H), 8.56 (d, J=8.4, 1H), 7.86 (dd, J=8.4, 4.1, 1H), 7.41 (dd, J=8.4, 5.7, 2H), 7.16, t, J=8.8, 2H), 4.60 (d, 6.3, 2H), 4.00-3.70 (m, 2H), 3.65-3.45 (m, 2H), 2.35-2.10 (m, 3H), 1.7 (m, 1H).

### Step 6: Sodium salt of 5-(1,1-Dioxido-1,2-thiazinan-2-yl)-N-(4-fluorobenzyl)-8-hydroxy-1,6-naphthyridine-7-carboxamide

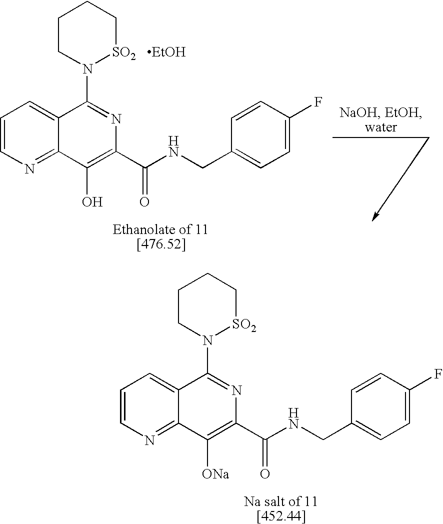

5-(N-1,4-Butanesultam)-N-(4-fluorobenzyl)-8-hydroxy-1,6-naphthyridine-7-carboxamide (11) monoethanolate (1.207 g, 2.533 mmol) was dissolved in a mixture of EtOH (24 mL) and water (11 mL) by heating to 78° C. for 1 h. A solution of 5M aq NaOH (0.608 mL, 3.04 mmol) was added over 15 min at 78° C. A yellow crystalline precipitate was deposited. The mixture was aged at 78° C. for 20 min, then cooled to 20° C. over 30 min and aged for 30 min at 20° C. The slurry was filtered and the filter cake was washed with 2:1 EtOH:water (5 mL) and EtOH (15 mL). The filter cake was dried under a stream of nitrogen to provide 1.088 g (95%) of 5-(N-1,4-butanesultam)-N-(4-fluorobenzyl)-8-hydroxy-1,6-naphthyridine-7-carboxamide sodium salt (11 sodium salt) as a yellow crystalline solid.

The Na salt was analyzed by differential scanning calorimetry at a heating rate of 10° C./min in an open cup under flowing nitrogen and was found to have a DSC curve exhibiting an endotherm with a peak temperature of about 348° C. and an associated heat of fusion of about 45 J/gm followed by an exotherm with a peak temperature of about 352° C. and an associated heat of fusion of about 45 J/gm.

The XRPD pattern of the Na salt was generated on a Philips Analytical X-ray powder diffraction (XRPD) instrument with XRG 3100 generator using a continuous scan from 2 to 40 degrees 2 theta over about 126 minutes. The resulting XRPD pattern was analyzed using Philips X'Pert Graphics and Identify software. Copper K-Alpha 1 radiation was used as the source. The experiment was run under ambient conditions. The XRPD pattern was found to have characteristic diffraction peaks corresponding to d-spacings of 12.63, 5.94, 5.05 4.94, 4.81, 4.61, 4.54, 4.34, 3.88, 3.73, 3.49, 3.45, 3.22, 3.15, 3.12, and 2.86 angstroms.

### EXAMPLE 4

### Preparation of 1,4-Butanesultam

Step 1

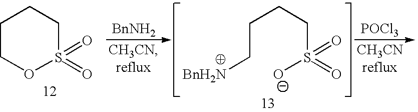

| | | | 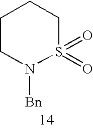 |
|---|---|---|---|
| | Amount | Moles | Equivalent |
| 1,4-Butane sultone | 68.10 g | 0.5000 | 1 |
| Benzylamine | 69.70 g | 0.6500 | 1.3 |
| Acetonitrile | 625 mL | | |
| Phosphorus oxychloride | 153.33 g | 1.000 | 2 |

A solution of 1,4-Butane sultone 12 (68.10 g, 0.5000 moles) and benzylamine (69.70 g, 0.6500 moles) in acetonitrile (625 mL) was reflux at 82° C. for 24 h. (The reaction was monitored by ¹H NMR until conversion of 12 to 13>98%). While the resulting slurry was cooled to 50° C., phosphorus oxychloride (153.33 g, 1.000 moles) was slowly added by dropping funnel. After complete addition, the mixture was reflux at 82° C. for 8 h. (The reaction was monitored by HPLC until conversion >98%) The reaction mixture was concentrated to remove most of acetonitrile. The residue was cooled to 0-5° C. and neutralized with 20% sodium hydroxide to pH=7. The resulting mixture was extracted with IPAc (3×350 mL). The combined extracts were washed with 10% sodium bicarbonate (2×100 mL) and 25% of brine (100 mL). The resulting clear solution was concentrated and solvent switched to methanol (total volume 1000 mL), which was used for next step reaction. For compound 14: ¹H NMR (CDCl₃, 400 MHz) δ: 7.38-7.32 (m, 5 H), 4.32 (s, 2H), 3.23 (m, 2 H), 3.11 (m, 2 H), 2.22 (m, 2 H), 1.62 (m, 2 H).

Step 2

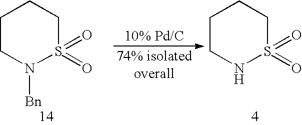

| | Amount | Moles | Equivalent |
|---|---|---|---|
| N-Benzyl-1,4-butanesultam | | 0.5000 | 1 |
| 10% Pd/C | 12.0 g | | 10% wt |
| 1 N HCl (aqueous) | 80 mL | | |
| Solka Fluck | 20 g | | |

To a solution of N-Benzyl-1,4-butanesultam 14 (0.5000 moles) in methanol (total volume 1000 mL) and 1 N HCl aqueous (80 mL) was added 10% Pd/C (12.0 g). The resulting slurry was submitted to hydrogenation at 40° C., 45 PSI for 24 h. (The reaction was monitored by HPLC until conversion of 14 to 4>99%). The reaction mixture was cooled to ambient temperature and filtered by passing through a pad of Solka Flock (20 g) and washed with methanol (3×100 mL). The combined filtrates were concentrated to remove the methanol. The crystalline solid was precipitated out during the concentration. To the slurry solution was added heptane/MTBE (3:2, 100 mL). The resulting mixture was cooled to 0° C., and aged for 0.5 h. The crystalline solid was filtered off and washed with cold heptane/MTBE (3:2, 50 mL, dried under vacuum with a nitrogen sweep to give 1,4-butanesultam 4 (49.8 g, 74% overall from 12).

While the foregoing specification teaches the principles of the present invention, with examples provided for the purpose of illustration, the practice of the invention encompasses all of the usual variations, adaptations and/or modifications that come within the scope of the following claims.

What is claimed is:

1. A process for preparing a compound of Formula (I): 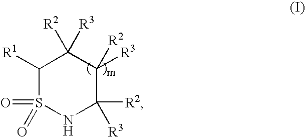
which comprises:
(B) treating a compound of Formula (II): 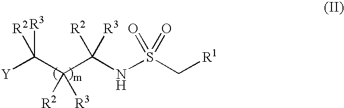
with a deprotonating agent in an aprotic solvent to obtain Compound I; wherein
Y is a leaving group without an active proton;
R¹ is H or C₁₋₆ alkyl;
each R² is independently H or C₁₋₆ alkyl;
each R³ is independently H or C₁₋₆ alkyl; and
m is an integer equal to zero, 1 or 2.

2. The process according to claim 1, which further comprises:
(A) reacting a sulfonyl halide of Formula (III) or a sulfonyl anhyride of Formula (IV): 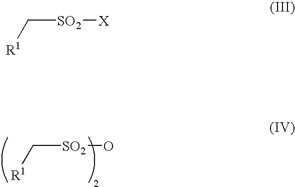
with an amine of Formula (V): 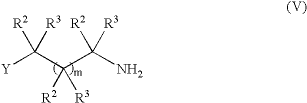
in an aprotic solvent to obtain Compound II; wherein X is halo.

3. The process according to claim 1, wherein R¹ is H or C₁₋₄ alkyl.

4. The process according to claim 1, wherein each R² is H and each R³ is H.

5. The process according to claim 1, wherein Y is
(1) halo,
(2) —OSO₂R^{a}, wherein R^{a} is phenyl which is optionally substituted with one or more substituents each of which is fluoro, chloro, methoxy, or t-butyl, or
(3) —OP(═O)(OR^{b})₂, wherein each R^{b} is independently a C₁₋₆ alkyl.

6. The process according to claim 5, wherein Y is halo.

7. The process according to claim 1, wherein Step B is conducted at a temperature in the range of from about −78 to about 50° C.

8. The process according to claim 1, wherein the deprotonating agent is selected from the group consisting of alkali metal salts and alkaline earth metal salts of di-C₁-C₉ alkylamines and C₄-C₉ cyclic secondary amines, alkali metal salts and alkaline earth metal salts of bis(tri-C₁-C₄ alkylsilyl)amines, alkali metal hydrides, alkali metal amides, C₁-C₁₀ alkyllithiums, C₆-C₁₀ aryllithiums, C₁-C₆ alkylmagnesium halides, and C₆-C₁₀ arylmagnesium halides.

9. The process according to claim 1, wherein the aprotic solvent is selected from the group consisting of hydrocarbons and ethers.

10. The process according to claim 1, wherein the deprotonating agent is employed in Step B in an amount in the range from about 1.1 to about 10 equivalents per equivalent of Compound II.

11. The process according to claim 2, wherein the reaction of Step A is conducted at a temperature in the range of from about −78 to about 50° C.

12. The process according to claim 2, wherein the aprotic solvent in Step A is selected from the group consisting of hydrocarbons, halogenated hydrocarbons, and ethers.

13. The process according to claim 2, wherein sulfonyl halide m or sulfonyl anhydride IV is employed in Step A in an amount in the range from about 0.2 to about 5 equivalents per equivalent of amine V.

14. A process for preparing 1,4-butanesultam 4: 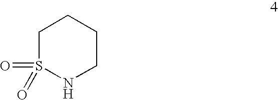
which comprises:
(bb) treating a compound of Formula (IIa): 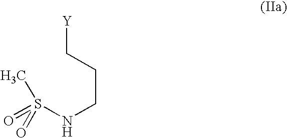
with a deprotonating agent in an aprotic solvent to obtain Compound 4; wherein Y is a leaving group without an active proton.

15. The process according to claim 14, which further comprises:
(aa) reacting a methanesulfonyl halide of Formula (IIIa) or methanesulfonic anhydride of Formula (IVa): 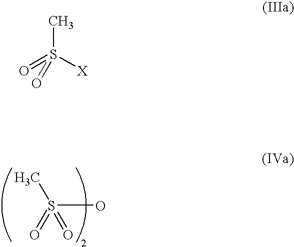
with an amine of Formula (Va): 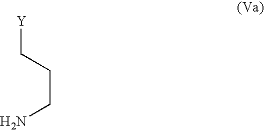
in an aprotic solvent to obtain Compound (IIa); wherein X is halo.

16. The process according to claim 14, wherein Y is
(1) halo,
(2) —OSO₂R^{a}, wherein R^{a} is phenyl which is optionally substituted with one or more substituents each of which is fluoro, chloro, methoxy, or t-butyl, or
(3) —OP(═O)(OR^{b})₂, wherein each R^{b} is independently a C₁₋₆ alkyl.

17. The process according to claim 16, wherein Y is halo.

18. The process according to claim 14, wherein
the treatment in Step (bb) is conducted at a temperature in the range of from about −78 to about 10° C.;
the deprotonating agent is an alkali metal salt of a di-C₁-C₉ alkylamine or a C₄-C₉ cyclic secondary amine;
the aprotic solvent is an ether; and
the deprotonating agent in Step (bb) is employed in an amount in the range from about 1.8 to about 3 equivalents per equivalent of Compound IIa.

19. The process according to claim 14, wherein Compound IIa is

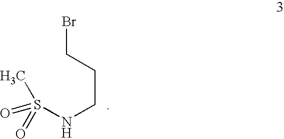

20. The process according to claim 15, wherein
the treatment in Step (bb) is conducted at a temperature in the range of from about −78 to about 10° C.;
the deprotonating agent is an alkali metal salt of a di-C₁-C₉ alkylamine or a C₄-C₉ cyclic secondary amine;
the aprotic solvent is an ether;
the deprotonating agent in Step (bb) is employed in an amount in the range from about 1.8 to about 3 equivalents per equivalent of Compound IIa.
the reaction in Step (aa) is conducted at a temperature in the range of from about −78 to about 30° C.
the aprotic solvent in Step (aa) is an ether, and
methanesulfonyl halide IIIa or methanesulfonic anhydride IVa is employed in Step (aa) in an amount in the range from about 0.5 to about 5 equivalents per equivalent of amine Va.

21. The process according to claim 20, wherein Compound IIa is 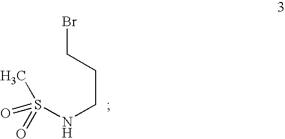
Compound IIIa is employed in Step (aa) and is

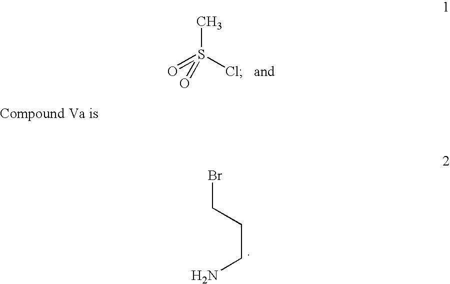

22. The process according to claim 15, which comprises:
(aa) reacting a methanesulfonyl halide of Formula (IIIa): 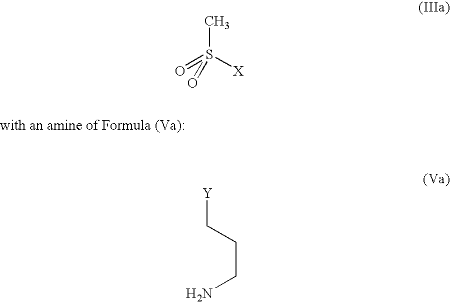
in an aprotic solvent to obtain a compound of Formula (IIa); 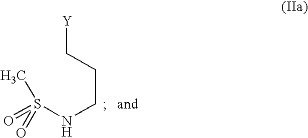
(bb) treating a Compound Ha with a deprotonating agent in an aprotic solvent to obtain Compound 4; wherein X and Y are each independently a halo.

23. The process according to claim 22, wherein:
the aprotic solvent in both Steps (bb) and (aa) is the same; and
the process is conducted without isolating Compound IIa from the reaction mixture at the conclusion of Step (aa).

24. The process according to claim 23, wherein:
the aprotic solvent in Steps (aa) and (bb) is an ether;
Compound IIa is

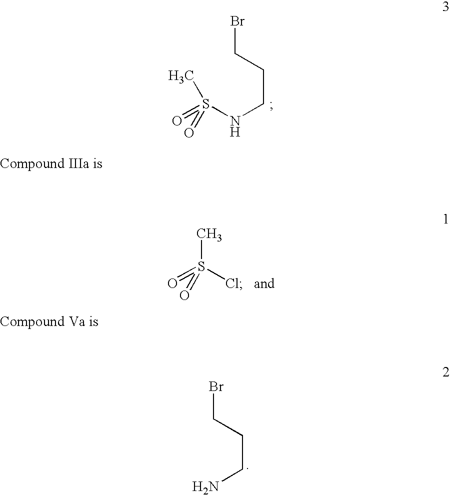

25. A process for preparing a compound of Formula (I): 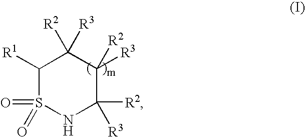
which comprises:
(A) substituting a compound of Formula (VII) 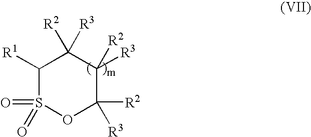
with benzylamine to form a slurry product, cooling the slurry product, and cyclizing the slurry product with a cyclizing agent to form a compound of Formula (VI): 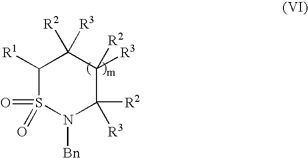
(B) deblocking the compound of Formula (VI) by hydrogenation in the presence of a Pd catalyst;
wherein
R¹ is H or C₁₋₆ alkyl;
each R² is independently H or C₁₋₆ alkyl;
each R³ is independently H or C₁₋₆ alkyl; and
m is an integer equal to zero, 1 or 2.

26. The process of claim 25, wherein the cyclizing agent is phosphorus oxychloride.

27. The process of claim 26, wherein m is 1, R¹ is hydrogen, R² is hydrogen and R³ is hydrogen.

28. The process of claim 27, wherein the Pd catalyst is Pd/C.
